(19) United States
(12) Patent Application Publication
(10) Pub. No.: US 2025/0206684 A1
(43) Pub. Date: Jun. 26, 2025

(54) PROCESS FOR CONVERTING NAPHTHA TO LIGHT PARAFFINS WITH RECYCLE

(71) Applicant: UOP LLC, Des Plaines, IL (US)

(72) Inventors: Scott Lyle Nauert, Chicago, IL (US); Ian G. Horn, Streamwood, IL (US); Roberto Rodriguez, Chicago, IL (US)

(21) Appl. No.: 18/967,227

(22) Filed: Dec. 3, 2024

Related U.S. Application Data
(60) Provisional application No. 63/613,038, filed on Dec. 20, 2023.

(51) Int. Cl. C07C 4/06; C07C 7/00; C07C 7/04

(52) U.S. Cl. CPC C07C 4/06; C07C 7/005; C07C 7/04

(57) ABSTRACT

A process for converting naphtha to ethane and propane is disclosed. The process comprises contacting a naphtha stream with a catalyst in a reactor to produce a converted stream. The converted stream is separated into a light paraffin stream and an aromatic stream. The aromatic stream is recycled to the reactor. The light paraffin stream is separated into an ethane stream and a propane stream.

### FIELD

The field is the conversion of naphtha to light paraffins. The field may particularly relate to converting naphtha to ethane and propane.

### BACKGROUND

Light olefin production is vital to the production of sufficient plastics to meet worldwide demand. Dehydrogenation is a process in which light paraffins such as ethane and propane can be dehydrogenated to make ethylene and propylene respectively, typically in the presence of a catalyst. Dehydrogenation can be achieved in either the presence of an oxidant such as oxygen or in the absence of an oxidant. Non-oxidative dehydrogenation is an endothermic reaction which requires external heat to drive the reaction to completion. Propane dehydrogenation (PDH) is a widely practiced example of non-oxidative dehydrogenation to produce propylene from propane. Ethane oxidative dehydrogenation is a newer oxidative process for converting ethane to ethylene which can be conducted at lower temperatures with lower carbon oxide emissions than non-oxidative and thermal cracking processes.

Fluid catalytic cracking (FCC) is another endothermic process that can be tuned to produce substantial propylene. However, not every FCC unit is tuned to make substantial propylene. Also, high propylene FCC units do not recover much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure, enhance olefin yield and reduce the formation and deposition of carbonaceous material in the cracking reactors. The process is therefore often referred to as steam cracking or pyrolysis.

Paraffins with a range of carbon numbers can be thermally cracked to produce olefins including ethane, propane, butanes, and naphtha. Ethane and naphtha feeds are typical due to higher light olefin yield than propane and butane feeds. Ethane feed is used in regions where light hydrocarbon gases are prevalent. In regions where gas is not abundant, naphtha feed is employed for steam cracking. Naphtha steam cracking has long set the price in the ethylene industry due to higher production cost versus ethane steam cracking. The world does not currently produce enough ethane to supply the growing demand for ethylene. Therefore, regions lacking ethane supply such as Asia and Europe rely mainly on naphtha steam cracking to supply ethylene. Naphtha steam cracking yields only about 30%-35% ethylene with the balance including both relatively high-value by-products comprising propylene, butadiene, and butene-1 and relatively low value by-products comprising pyoil, pygas, and fuel gas. Additional pressures on naphtha steam cracking including minimum production requirements and environmental concerns have led to the withholding of government approvals in certain regions such as China. The ethylene industry needs a more efficient, economical and environmentally friendly route to light olefins from naphtha feeds.

### BRIEF SUMMARY

A process for converting naphtha to light paraffins is disclosed. The process comprises contacting a naphtha stream with a catalyst in a reactor to produce a converted stream. An aromatics recycle stream may be separated from the converted stream. The aromatics recycle stream may be recycled back to the reactor. An apparatus for converting naphtha is also disclosed. The apparatus comprises a plurality of reactors, wherein each reactor of the plurality of reactors is in fluid communication with a naphtha feed line. The apparatus also comprises a fractionation column in downstream fluid communication with the plurality of reactors. The fractionation column is provided with an upper reboiler and a lower reboiler to optimize the heat duty of the overall apparatus and the process for converting naphtha.

The process may provide a more efficient, economical and environmentally friendly route to light olefins from naphtha feeds. The process provides converting the naphtha to light paraffin feeds such as ethane and propane. The light paraffin feeds may be converted to olefins through more selective technologies such as propane dehydrogenation, ethane steam cracking, and ethane oxidative dehydrogenation.

### BRIEF DESCRIPTION OF THE DRAWINGS

FIG. 1 1 is a schematic drawing of a process and apparatus in accordance with an embodiment of the present disclosure.

FIG. 2 is a schematic drawing of a process and apparatus in accordance with another embodiment of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx−” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take the main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

C3-C8 hydrocarbon feed stock is primarily charged to a “Naphtha to Ethane and Propane” (NEP) unit to convert naphtha in the presence of hydrogen into desirable ethane and propane along with less desirable methane. “Naphtha to ethane and propane” is a highly exothermic process. Without management of the exotherm, the temperature may become unacceptably high leading to inoperability of the process and poor yields. The negative impacts of high temperature include rapid catalyst deactivation, low selectivity to ethane and propane, high selectivity to undesirable byproducts such as methane and aromatics, high rate of coke formation, and metallurgical concerns such as mechanical strength and coking tendency.

The present disclosure provides a process and an apparatus for converting naphtha to ethane and propane. The process comprises separating an NEP reactor converted stream into a product gas stream, an aromatics diluent recycle stream, and an aromatics product stream. The benefits of separating and recycling the aromatics diluent stream comprise improved exotherm management in the reactors, safety via a shorter process control loop, and reduced equipment count. The benefit of separating the aromatics diluent recycle stream separately from the aromatics product stream is to enable utilization of a lower grade utilities than the process and apparatus without producing separate aromatic diluent recycle and aromatic product streams.

The chemistry of NEP process is exothermic, requiring temperature management to prevent runaway reactions. This can include removing heat from the reactor or splitting feed between multiple reactors to manage heat release. An aromatics-rich stream may be used as an inert diluent to absorb heat in the multiple reactors. However, the aromatics diluent stream must be separated from the NEP reactor converted and recycled to the NEP reactors. The aromatic diluent stream should be separated with as little capital and utilities as possible while maintaining a short recycle path for responsive process control. The present disclosure discloses separating the aromatic diluent stream from a light paraffin stream and aromatics product first.

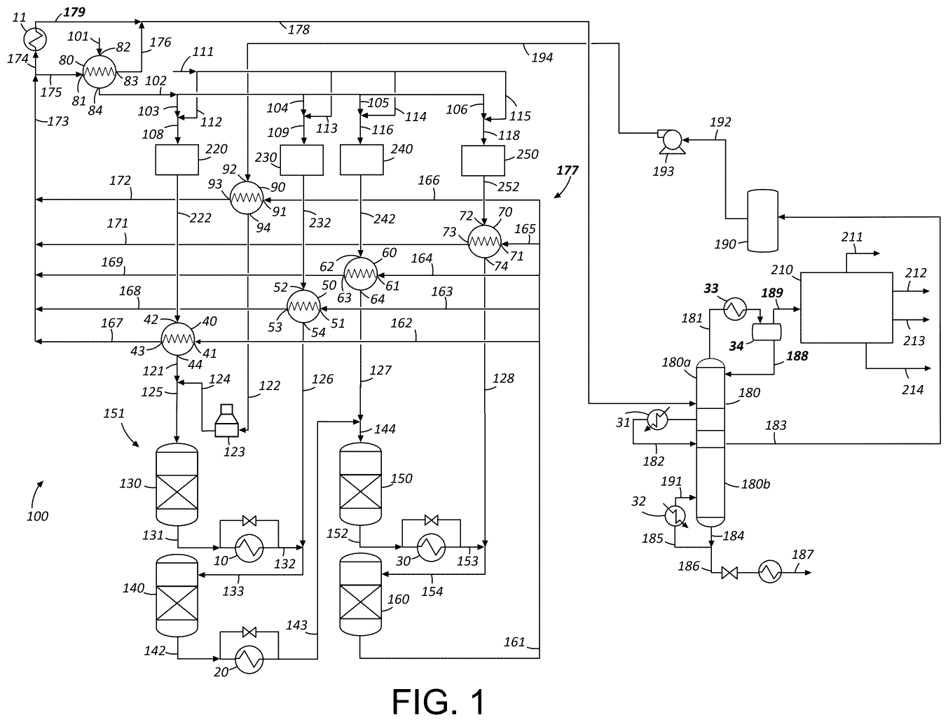

Turning to FIG. 1, an embodiment of a process and an apparatus for converting naphtha to ethane and propane 100 is disclosed. The process and apparatus 100 comprise an NEP reactor 151, a heat exchanger system 177, a splitter column 180, and a separation unit 210. In an aspect, the NEP reactor 151 may comprise a multistage NEP reactor. In an exemplary embodiment shown in the FIG. 1, the NEP reactor 151 may comprise four reactors, a first NEP reactor 130, a second NEP reactor 140, a third NEP reactor 150, and a fourth NEP reactor 160. A naphtha stream in line 102 taken from the main naphtha line 101 may be combined with a hydrogen stream in line 111 to provide a charge stream which is passed to the NEP reactor 151. Each of the first NEP reactor 130, the second NEP reactor 140, the third NEP reactor 150, and the fourth NEP reactor 160 are in fluid communication with the naphtha feed line 102. In an exemplary embodiment, the naphtha stream in line 102 may be a preheated naphtha stream. In accordance with the present disclosure, the naphtha stream in line 102 may be separated into a plurality of naphtha streams each passed to a dedicated NEP reactor of the multistage reactor 151. In an exemplary embodiment, the naphtha stream in line 102 may be separated into four naphtha streams a first naphtha stream in line 103, a second naphtha stream in line 104, a third naphtha stream in line 105, and a fourth naphtha stream in line 106. In another aspect, a hydrogen stream in line 111 may be separated into a plurality of hydrogen streams to provide a dedicated hydrogen stream for each of the naphtha stream and the NEP reactors. In accordance with another exemplary embodiment, the hydrogen stream in line 111 may be separated into four hydrogen streams, a first hydrogen stream in line 112, a second hydrogen stream in line 113, a third hydrogen stream in line 114, and a fourth hydrogen stream in line 115. The dedicated hydrogen streams may be combined with the dedicated naphtha streams to provide a dedicated charge stream for each of the plurality of reactors.

As shown in FIG. 1, the first naphtha stream in line 103 may be combined with the first hydrogen stream in line 112 to provide a dedicated first charge stream in line 108. The first charge stream in line 108 is passed to the dedicated first reactor 130. In an aspect, the dedicated first charge stream in line 108 may be passed to the dedicated first reactor 130 at a pressure of about 2068 kPa (300 psig) to about 2758 kPa (400 psig) or about 2137 kPa (310 psig) to about 2482 kPa (360 psig). Similarly, the second naphtha stream in line 104 may be combined with the second hydrogen stream in line 113 to provide a dedicated second charge stream in line 109, the third naphtha stream in line 105 may be combined with the third hydrogen stream in line 114 to provide a dedicated third charge stream in line 116, and the fourth naphtha stream in line 106 may be combined with the fourth hydrogen stream in line 115 to provide a dedicated fourth charge stream in line 118. In an aspect, the first charge stream in line 108, the second charge stream in line 109, the third charge stream in line 116, and the fourth charge stream in line 118 may be heated in the heat exchanger system 177 by heat exchange with a converted stream before it is charged to the dedicated reactor. In an embodiment, the heat exchanger system 177 may comprise a plurality of dedicated heat exchangers for heating each of the dedicated naphtha streams or the dedicated charge streams. In an exemplary embodiment, the heat exchanger system 177 may comprise four dedicated heat exchangers, a first heat exchanger 40, a second heat exchanger 50, a third heat exchanger 60, and a fourth heat exchanger 70.

The first charge stream in line 108 is passed to the first heat exchanger 40 to provide a first heated charge stream in line 121. In an exemplary embodiment, the first charge stream in line 108 may be passed to a first dedicated vaporizer 220 to vaporize some or all of the first charge stream in line 108. A vaporized first charge stream is taken in line 222 and passed to the first heat exchanger 40. The first heat exchanger 40 may comprise a hot inlet side 41 and a hot outlet side 43 for the hot converted stream in line 162, and a cold inlet side 42 and a cold outlet side 44 for the cold vaporized first charge stream in line 222. A hot converted stream is passed to the hot inlet side 41 and after heat exchange is removed from the hot outlet side 43 of the first heat exchanger 40. The vaporized first charge stream in line 222 is passed to the cold inlet side 42 and after heat exchange, the first heated charge stream is removed from the cold outlet side 44 of the first heat exchanger 40 in line 121. As described herein after in detail, the first heated charge stream in line 121 may be combined with a recycle stream in line 124 to provide a first combined charge stream in line 125. The first combined charge stream in line 125 is passed to the first NEP reactor 130. The first naphtha stream and the first hydrogen stream are contacted with an NEP catalyst in the first NEP reactor 130 to provide a first converted stream. The first NEP reactor 130 is in fluid communication with a branch naphtha line 103 of the naphtha feed line 102.

In accordance with the present disclosure, the naphtha stream may comprise C4 to C12 hydrocarbons preferably having a T10 between about −10° C. and about 60° C. and a T90 between about 70 and about 180° C. The naphtha feed stream may comprise normal paraffins, iso-paraffins, olefins, naphthenes, and aromatics. The naphtha stream may be heated to a reaction temperature of about 300° C. to about 600° C., suitably between about 325° C. and about 550° C., and preferably between about 350° C. and about 525° C. Overall weight hourly space velocity, defined herein as hourly mass flow rate of feed in stream 102 divided by total catalyst in all reactors should be between about 0.3 to about 20 hr⁻¹, suitably between about 0.5 and about 10 hr⁻¹ and preferably between about 1 to about 4 hr⁻¹. A total pressure should be about 0.1 to about 3 MPa (abs), or from about 1.5 to about 2.5 MPa (abs), preferably greater than 1 MPa (abs). In an embodiment, the first NEP reactor 130 may be operated at a higher pressure than the other reactors. Under these conditions, C2-C4 yield is consistently in an excess of 80 wt %, while methane yield is less than about 16 wt %, suitably below about 14 wt % and typically below about 12 wt % and preferably no more than 10 wt %.

In accordance with the present disclosure, the first NEP reactor 130, the second NEP reactor 140, the third NEP reactor 150, and the fourth NEP reactor 160 may be operated at similar or different operating conditions including reactor temperature and pressure.

The hydrogen-to-hydrocarbon molar ratio is important to selectively producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200% of stoichiometric requirements to convert naphtha molecules to ethane and/or propane.

The molar ratio of hydrogen to hydrocarbon depends on the feed type including paraffin, olefin, naphthene or aromatics, the feed molecular carbon number, and the desired product between predominantly ethane, predominantly propane, or ethane and propane of comparable abundance. For example, converting 1 mole of propane to ethane at stoichiometry, the process would require co-feeding 0.5 moles of hydrogen. In practice, the process can operate above or below this stoichiometry of 0.5 such as 0.33 to achieve greater than 40% ethane and less than 15% methane, depending on the process design parameters such as, feed contaminants, reactor type (fixed bed, moving bed, fluidized bed), and regeneration frequency. As the carbon number of feed molecules increases from light naphtha (C₄-C₇) to full range naphtha (C₆-C₁₀) the amount of hydrogen required for the reaction increases. For example, it would require 3.5 moles hydrogen and 2.0 moles of hydrogen to fully convert 1 mole of nonane to ethane and propane, respectively. The disclosed process can operate at three to five times the hydrogen-to-hydrocarbon ratio required to stoichiometrically convert the feed molecules to ethane and propane, respectively. It can also operate at 50% of hydrogen-to-hydrocarbon ratio required to stoichiometrically convert the feed molecules to ethane and propane, respectively. The hydrogen-to-hydrocarbon ratio would also depend on the need to produce petrochemical aromatics such as benzene, toluene and xylene.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce ⅛-inch to about 1/32-inch diameter calcined supports. Alternatively, the zeolite may be mixed with a silicon oxide containing binder and extruded to 1/32 to ¼ inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each of the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steamed oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 300° C. to about 550° C. for 3 hours before contacting feed.

In accordance with the present disclosure, the first NEP reactor 130, the second NEP reactor 140, the third NEP reactor 150, and the fourth NEP reactor 160 may comprise similar or different NEP catalysts.

After conversion, a first converted stream is discharged from the first NEP reactor 130 in line 131. The first converted stream in line 131 may be passed to a heat exchanger 10 to cool the stream, perhaps generating steam. A cooled first converted stream is taken in line 132 from the heat exchanger 10 and passed to the second NEP reactor 140. Alternatively, the heat removal between the stages may include producing superheated steam or heating of another process stream within or outside the current process. The second charge stream in line 109 is also passed to the second NEP reactor 140. The second charge stream in line 109 may be heated in the heat exchanger system 177 to provide a heated second charge stream in line 126 which is passed to the second NEP reactor 140. In an exemplary embodiment, the second charge stream in line 109 is passed to the second heat exchanger 50 to provide a second heated charge stream in line 126. In an exemplary embodiment, the second charge stream in line 109 may be passed to a second dedicated vaporizer 230 to vaporize some or all of the second charge stream in line 109. A vaporized second charge stream is taken in line 232 and passed to the second heat exchanger 50. The second heat exchanger 50 may comprise a hot inlet side 51 and a hot outlet side 53 for the hot converted stream, and a cold inlet side 52 and a cold outlet side 54 for the cold vaporized second charge stream. A hot converted stream is passed to the hot inlet side 51 and after heat exchange is removed from the hot outlet side 53 of the second heat exchanger 50. The vaporized second charge stream in line 232 is passed to the cold inlet side 52 and after heat exchanger, the second heated charge stream is removed from the cold outlet side 54 of the second heat exchanger 50 in line 126.

In an exemplary embodiment, the heat exchanged second charge stream in line 126 may be combined with the heat exchanged first converted stream in line 132 to provide a combined second charge stream in line 133 which is passed to the second NEP reactor 140. In the second NEP reactor 140, the first converted stream and the second charge stream are contacted with an NEP catalyst to provide a second converted stream in line 142. The NEP catalyst in the second NEP reactor 140 can be similar or a different NEP catalyst than the first NEP reactor 130. The second NEP reactor 140 is in fluid communication with a branch naphtha line 104 of the naphtha feed line 102.

After conversion, the second converted stream is taken in line 142 from the second NEP reactor 140. The second converted stream in line 142 may be passed to a heat exchanger 20 to generate steam. A heat exchanged perhaps cooled second converted stream is taken in line 143 from the heat exchanger 20 and passed to the third NEP reactor 150. Alternatively, the heat removal between the stages may be via superheating of steam or heating of another process stream within or outside the current process. The third charge stream in line 116 is also passed to the third NEP reactor 140. The third charge stream in line 116 may be heated in the heat exchanger system 177 to provide a heat exchanged perhaps heated third charge stream in line 127 which is passed to the third NEP reactor 150. In an exemplary embodiment, the third charge stream in line 116 is passed to the third heat exchanger 60 to provide a third heated charge stream in line 127. In an exemplary embodiment, the third charge stream in line 116 may be passed to a third dedicated vaporizer 240 to vaporize some or all of the third charge stream in line 116. A vaporized third charge stream is taken in line 242 and passed to the third heat exchanger 60. The third heat exchanger 60 may comprise a hot inlet side 61 and a hot outlet side 63 for the hot converted stream, and a cold inlet side 62 and a cold outlet side 64 for the cold vaporized third charge stream. A hot converted stream in line 164 is passed to the hot inlet side 61 and after heat exchange is removed from the hot outlet side 63 of the third heat exchanger 60. The vaporized third charge stream in line 242 is passed to the cold inlet side 62 and after heat exchange, the third heated charge stream is removed from the cold outlet side 64 of the third heat exchanger 60 in line 127.

In an exemplary embodiment, the third heated charge stream in line 127 may be combined with the heat exchanged second converted stream in line 143 to provide a combined third charge stream in line 144 which is passed to the third NEP reactor 150. In the third NEP reactor 150, the second converted stream and the third charge stream are converted with a NEP catalyst to provide a third converted stream in line 152. The NEP catalyst in the third NEP reactor 150 can be similar or a different NEP catalyst than the first NEP reactor 130 and the second NEP reactor 140. The third NEP reactor 150 is in fluid communication with a branch naphtha line 105 of the naphtha feed line 102.

After conversion, the third converted stream is taken in line 152 from the third NEP reactor 140. The third converted stream in line 152 may be passed to a heat exchanger 30 to generate steam. A heat exchanged perhaps cooled third converted stream is taken in line 153 from the heat exchanger 30 and passed to the fourth NEP reactor 160. Alternatively, the heat removal between the stages may be via superheating of steam or heating of another process stream within or outside the current process. The fourth charge stream in line 118 is also passed to the fourth NEP reactor 160. The fourth charge stream in line 118 may be heated in the heat exchanger system 177 to provide a heat exchanged perhaps heated fourth charge stream in line 128 which is passed to the fourth NEP reactor 160. In an exemplary embodiment, the fourth charge stream in line 118 is passed to the fourth heat exchanger 70 to provide a fourth heated charge stream in line 128. In an exemplary embodiment, the fourth charge stream in line 118 may be passed to a fourth dedicated vaporizer 250 to vaporize some or all of the fourth charge stream in line 118. A vaporized fourth charge stream is taken in line 252 and passed to the fourth heat exchanger 70. The fourth heat exchanger 70 may comprise a hot inlet side 71 and a hot outlet side 73 for the hot converted stream, and a cold inlet side 72 and a cold outlet side 74 for the cold vaporized fourth charge stream in line 252. A hot converted stream in line 165 is passed to the hot inlet side 71 and after heat exchange is removed from the hot outlet side 73 of the fourth heat exchanger 70. The vaporized fourth charge stream in line 252 is passed to the cold inlet side 72 and after heat exchange, the fourth heated charge stream is removed from the cold outlet side 74 of the fourth heat exchanger 70 in line 128.

In an exemplary embodiment, the heat exchanged fourth charge stream in line 128 may be combined with the heat exchanged third converted stream in line 153 to provide a combined fourth charge stream in line 154 which is passed to the fourth NEP reactor 160. In the fourth NEP reactor 160, the third converted stream and the fourth charge stream are converted with an NEP catalyst to provide a fourth converted stream in line 161. The NEP catalyst in the fourth NEP reactor 160 can be similar or a different NEP catalyst than the first NEP reactor 130, the second NEP reactor 140, and the third NEP reactor 160. The fourth NEP reactor 160 is in fluid communication with a branch naphtha line 106 of the naphtha feed line 102. In accordance with the present disclosure, the fourth converted stream in line 161 may be a last stage or reactor converted stream.

The present disclosure also includes preheating both the naphtha stream in the main naphtha line 101 and the dedicated naphtha streams before charging them to their dedicated NEP reactors. In accordance with the present disclosure, naphtha stream in the main naphtha line 101, the first naphtha stream in line 103, the second naphtha stream in line 104, the third naphtha stream in line 105, and the fourth naphtha stream in line 106 are heat exchanged with a converted stream from the last NEP reactor of the multistage NEP reactor 151.

After paraffin conversion, a light paraffin stream is discharged from the fourth NEP reactor 160 in the fourth converted stream in line 161. In an aspect, the fourth converted stream in line 161 may be separated into a plurality of streams for heating the dedicated charge streams in the heat exchanger system 177. In an exemplary embodiment, the fourth converted stream in line 161 may be separated into five hot streams, a first hot converted stream in line 162, a second hot converted stream in line 163, a third hot converted stream in line 164, a fourth hot converted stream in line 165, and a fifth hot converted stream in line 166. The first hot converted stream in line 162 is passed to the hot inlet side 41 of the first heat exchanger 40 to heat the vaporized first charge stream in line 222. A cooled but substantially hot first converted stream is removed from the hot outlet side 43 of the first heat exchanger 40 in line 167. The second hot converted stream in line 163 is passed to the hot inlet side 51 of the second heat exchanger 50 to heat the vaporized second charge stream in line 232. A cooled but substantially hot second converted stream is removed from the hot outlet side 53 of the second heat exchanger 50 in line 168. The third hot converted stream in line 164 is passed to the hot inlet side 61 of the third heat exchanger 60 to heat vaporized third charge stream in line 242. A cooled but substantially hot third converted stream is removed from the hot outlet side 63 of the third heat exchanger 60 in line 169. The fourth hot converted stream in line 165 is passed to the hot inlet side 71 of the fourth heat exchanger 70 to heat the vaporized fourth charge stream in line 252. A cooled but substantially hot fourth converted stream is removed from the hot outlet side 73 of the fourth heat exchanger 70 in line 171. The cooled first converted stream in line 167, the cooled second converted stream in line 168, the cooled third converted stream in line 169, and the cooled fourth converted stream in line 171 may be combined to provide a cooled but substantially hot/warm light paraffin stream in line 173.

The cooled light paraffin stream in line 173 is substantially hot for further separation. The cooled light paraffin stream in line 173 may be used to further heat or provide heat to another process stream or a reboiler. In an embodiment, the cooled light paraffin stream in line 173 may be separated into a first light paraffin stream in line 174 and a second light paraffin stream in line 175. The first light paraffin stream in line 174 may be passed through a heat exchanger 11 to release heat and produce steam or heat a process stream. In an exemplary embodiment, the first light paraffin stream in line 174 may be passed through a column reboiler 11 to provide heat duty to the column reboiler 11. A cooled first light paraffin stream is taken in line 179 from the column reboiler 11.

The second light paraffin stream in line 175 may be taken and heat exchanged with a naphtha stream in a fifth heat exchanger 80. In an exemplary embodiment, the second light paraffin stream in line 175 may be passed to the fifth heat exchanger 80 to heat the naphtha stream in the main naphtha line 101 to provide the preheated naphtha stream in line 101. The fifth heat exchanger 80 may comprise a hot inlet side 81 and a hot outlet side 83 for the hot second light paraffin stream in line 175, and a cold inlet side 82 and a cold outlet side 84 for the cold naphtha stream in line 101. The second light paraffin stream in line 175 is passed to the hot inlet side 81 and after heat exchange the cooled second light paraffin stream is removed from the hot outlet side 83 of the fifth heat exchanger 80 in line 176. The naphtha stream in the main naphtha line 101 is passed to the cold inlet side 82 and after heat exchange, the preheated naphtha stream is removed from the cold outlet side 84 of the fourth heat exchanger 70 in line 102. The preheated naphtha stream in line 102 is separated into the dedicated naphtha streams 103 to 106.

The cooled first light paraffin stream in line 179 and the cooled second light paraffin stream in line 176 are combined to provide a combined light paraffin stream in line 178. The combined light paraffin stream in line 178 may be separated to provide a C4− stream in line 189 and one or more aromatics streams. The C4− stream may comprise at least about 40 wt % ethane or at least about 40 wt % propane or at least about 70 wt % and preferably at least 80 wt % ethane and propane. The ethane to propane ratio can range from about 0.1 to about 5. The C4− stream can have less than about 16 wt %, suitably less than about 14 wt %, preferably less than about 12 wt %, and more preferably less than about 10 wt % methane.

The combined light paraffin stream in line 178 may be fed to the splitter column 180. The splitter column 180 is in downstream fluid communication with the plurality of reactors, the second NEP reactor 140, the third NEP reactor 150, and the fourth NEP reactor 160. In an aspect, the splitter column 180 is a fractionation column that separates ethane and propane from aromatics. In an exemplary embodiment, the combined light paraffin stream in line 178 may be fed to the splitter column 180 at a temperature of about 100° C. (212° F.) to about 200° C. (392° F.), and a pressure of about 1378 kPa (gauge) (200 psig) to about 2068 kPa (gauge) (300 psig). The splitter column 180 may comprise an upper section 180a and a lower section 180b. The combined light paraffin stream in line 178 may be fed to the splitter column 180 at a location to the upper section 180a of the splitter column 180. The splitter column 180 produces an overhead stream 181, an aromatic product stream 186, and an aromatic recycle side stream 183 to be described below. In an exemplary embodiment, the splitter column 180 may be operated at an overhead pressure of about 1378 kPa (gauge) (200 psig) to about 2068 kPa (gauge) (300 psig) and an overhead temperature of about 60° C. (140° F.) to about 200° C. (392° F.).

The overhead stream in line 181 may comprise hydrogen, C4− hydrocarbons and some aromatics. The aromatics must be separated from the overhead stream in line 181 to lower the heat duty and utilities for further separation of ethane and propane from the overhead stream. In an aspect of the present disclosure, the overhead stream in line 181 may be cooled in a condenser 33 and passed to condenser receiver 34. A liquid stream 188 comprising aromatics is taken from the condenser receiver 34 and recycled to the splitter column 180 as reflux. A vapor stream 189 comprising C4− hydrocarbons is taken from the condenser receiver and passed to the NEP separation unit 210 to separate ethane and propane product streams.

The NEP separation unit 210 may be a fractionation column or a series of fractionation columns and other separation units that may separate the overhead stream in line 181 into the hydrogen stream in line 211, an ethane stream in line 212, a propane stream in line 213, and a C4+ stream in line 214.

The NEP separation unit 210 may comprise a compressor to increase pressure suitable for downstream separation. The NEP separation unit 210 may comprise a demethanizer column that separates the overhead light paraffin stream into a gas stream in an overhead line and a C2+ paraffin stream in a bottoms line. The gas stream may be sent to a hydrogen purification unit such as a PSA unit to recover hydrogen in line 211 for recycle to the NEP reactor 151. Remaining methane from the hydrogen purification unit may be used for fuel gas. The C2+ paraffin stream may then be fed to a deethanizer column to produce the ethane stream in a deethanizer overhead line 212 and a C3+ paraffin stream in a deethanized bottoms line.

The C3+ paraffin stream may then be fed to a depropanizer column to produce the propane stream in a depropanizer overhead line 213 and the heavy paraffin stream in line 214 which may comprise C4+ hydrocarbons.

The aromatics are separated from the reflux stream in line 188 in the splitter column 180. The present process separates aromatics into an aromatic recycle stream and aromatics product stream. In an aspect of the present disclosure, the aromatic recycle stream may be separated and taken as liquid side stream from the splitter column 180 in line 183. In an exemplary embodiment, the aromatic recycle stream in line 183 predominantly comprises single ring aromatics comprising benzene, toluene, and xylenes. The aromatic recycle stream in line 183 may be taken from a location from the top half of the lower section 180b of the splitter column 180. In an embodiment, the ratio of aromatic recycle stream in line 183 to total naphtha stream in line 102 may comprise 0.1 to 1.0 by weight. In accordance with the present disclosure, the aromatic recycle stream in line 183 may comprise greater than 90 wt % single ring aromatics and greater than 95 wt % aromatics. To optimize utility levels for separation, the splitter column 180 may comprise an upper reboiler 31 and a lower reboiler 32. A side reboiler stream is also taken from the upper section 180a of the splitter column 180, passed through the upper reboiler 31 and recycled to the bottom section 180b of the splitter column 180 in line 182. The upper reboiler 31 may be heated with a high-pressure steam. In an exemplary embodiment, the side reboiler stream may be heated to a temperature of about 150° C. (302° F.) to about 250° C. (482° F.) in the upper reboiler 31.

In an alternate embodiment, the overhead stream in line 181 may be passed through a reboiler (not shown) to reboil a column in NEP separation unit 210 before it is passed to condenser 33.

Referring to the splitter column 180, the upper reboiler 31 may be located below the feed tray for the feed stream in line 178 to the splitter column 180 in the upper section 180a and above the aromatics recycle stream in line 183 of the splitter column 180 in the lower section 180b. The splitter column 180 may also comprise one or more liquids accumulator trays. The accumulator trays collect all or a portion of the liquid for feeding the upper reboiler and the aromatics recycle stream 183. The bottoms stream from the splitter column 180 has a normal vapor pressure which may be sufficiently low for storage. In accordance with the present disclosure, the upper reboiler 31 is heated by steam and the bottoms reboiler 32 is heated with hot oil. In an exemplary embodiment, the splitter column 180 may be operated at a column pressure of greater than 15 bar(a) or greater than 10 bar(a). The C4− hydrocarbons need to be separated from the aromatics in the splitter column 180 to generate ethane and propane feeds for downstream processes. The separation of aromatics from C4− hydrocarbons typically requires high pressure supplied by a compressor. There is significant benefit to operating the splitter column at high pressure to minimize downstream compression. The splitter column 180 may be operated at a pressure that matches with the NEP reactor converted pressure in line 178. The aromatics recycle stream may be recycled back to the NEP reactor 151 as inert diluent to help manage the reactor exotherm that may generate. Higher aromatics purity is preferable because aromatics are inert in the NEP reactor so higher purity improves the ability to manage heat in the reactor 151. C4− hydrocarbons do not produce significant heat upon recycle, so it may be acceptable to have some amount of C4− hydrocarbons dissolved in the recycle aromatics. In an aspect, the aromatics recycle stream 183 may be taken from the side of the splitter column below the feed tray and below the upper reboiler 31 of the splitter column. In accordance with the present disclosure, the purity of the aromatics side stream 183 is set by operation of the upper reboiler 21. The upper reboiler 31 preferably is heated with steam. The side line 183 for the aromatics recycle stream is in fluid communication with the multistage reactor 151. The temperature required to meet the purity specification is set by the splitter column pressure.

Depending on the splitter column pressure, the upper reboiler 31 may be heated by a stream which is high pressure steam. In an embodiment, the steam to the upper reboiler 31 may be supplied at a pressure of about greater than 30 bar(a). The NEP reactor 151 intercoolers generate high pressure steam, so there is a significant utilities advantage to using high pressure steam for the upper reboiler 32. The aromatic bottoms product stream from the splitter column 180 has sufficiently low vapor pressure for storage at atmospheric pressure. This purity requirement and the column pressure determine the temperature required in the lower reboiler. Generally, this requires higher temperature than can be supplied with steam, and so the bottoms reboiler 32 must be heated with hot oil or fired heat. Hot oil is preferable to a fired heater because the lower reboiler duty is very small, and a hot oil package is more economic for small scale.

The bottoms aromatics stream is taken in bottoms line 184 from the splitter column 180. A bottoms reboil stream is taken from the bottoms stream in line 185, passed through a bottoms reboiler 32 and recycled back to the splitter column 180 in a reboiled line 191. The bottoms reboiler 32 may be heated with hot oil or fired heat. In an exemplary, the bottoms reboil stream may be heated to a temperature of about 200° C. (392° F.) to about 350° C. (662° F.) in the bottoms reboiler 32. An aromatics product stream is taken in line 186 from the bottoms of the splitter column 180. In accordance with the present disclosure, the aromatic product stream in line 186 may comprise greater than 95 wt % aromatics. The aromatic product stream may be sent to storage in line 187.

In accordance with the present disclosure, the aromatics product stream has a lower vapor pressure requirement than the aromatics recycle stream. Excess C4− hydrocarbons are stripped from the column bottoms aromatics product stream to meet the aromatics product purity requirement. The process separates C4− gases, an aromatics-rich recycle stream, and a high purity aromatics product in a single distillation column using a minimal amount of fired heat or hot oil. The current process provides the shortest control loop for providing a safety and process control advantage. The disclosure also allows for the lowest reboiler temperature in columns separating C4− hydrocarbons, allowing for more heat integration throughout the NEP process.

The aromatics recycle stream is taken in line 183 and passed to a surge drum 190. The surge drum 190 is provided on the aromatics recycle stream in line 183 to provide process control. The surge drum 190 may be sized to provide sufficient surge time to be able to respond to changes in process conditions. From the drum 190, the aromatics recycle stream is taken in line 192 and passed to the NEP reactor 151. In an aspect, the aromatics recycle stream may be pumped to the NEP reactor 151 via a pump 193 in line 194. The aromatics recycle stream in line 194 may be heat exchanged with the converted stream in the heat exchanger system 177 to provide a preheated aromatics recycle stream in line 122. In an embodiment, the heat exchanger system 177 may comprise six dedicated heat exchangers. In an exemplary embodiment, the heat exchanger system 177 comprises a sixth dedicated heat exchanger 90 for heating the aromatics recycle stream in line 194. The sixth heat exchanger 90 may comprise a hot inlet side 91 and a hot outlet side 93 for the fifth hot converted stream in line 166, and a cold inlet side 92 and a cold outlet side 94 for the aromatics recycle stream in line 194. The aromatics recycle stream in line 194 may be heated with a converted stream in the sixth heat exchanger 90. In an embodiment, the fifth hot converted stream in line 166 is passed to the hot inlet side 91 of the sixth heat exchanger 90. A cooled but substantially warm/hot fifth converted stream is removed from the hot outlet side 93 of the sixth heat exchanger 90 in line 172. The cooled fifth converted stream in line 172 is combined with the other four heat exchanged converted streams to provide the cooled light paraffin stream in line 173.

A preheated aromatics recycle stream is removed from the cold outlet side 94 of the sixth heat exchanger 90 in line 122. The preheated aromatics recycle stream in line 122 may be further heated in a fired heater 123 to provide a heated aromatics recycle stream in line 124. The heated aromatics recycle stream in line 124 may be passed to the first NEP reactor in line 125 and to absorb the exotherm generated in the NEP reactors as previously described. The aromatics stream passes through all of the NEP reactors in series. In an exemplary embodiment, the ratio of the mass rate of the aromatics recycle stream and the first naphtha stream in the first combined charge stream in line 125 may range from about 0.1 to about 1.

In an aspect of the present disclosure, the heat exchanger system may comprise a combined heat exchanger for heating the dedicated charge streams in a single heat exchanger.

Referring back to the splitter column 180, the NEP separation unit 210 may take other forms. For example, the NEP separation unit 210 may omit a demethanizer column and the overhead light paraffin stream in line 181 may feed a deethanizer column which produces a C2− stream in a deethanizer overhead line. The C2− stream can be separated in the hydrogen purification unit to recover a hydrogen stream in line 211 while residual ethane and methane from the hydrogen purification unit can comprise or supplement the ethane stream in line 212. The hydrogen purification unit may comprise a membrane unit and the hydrogen recovered from the membrane unit may be further purified in an absorption column before it is recycled to the NEP reactor 151 in line 111. In an additional alternative, the C2− stream from the deethanizer column may be charged to an ethylene producing unit (not shown) in which ethane is converted to ethylene but methane and hydrogen rides through inertly to be recovered in a downstream ethylene recovery unit.

The ethane stream in line 212 may be charged to an ethylene producing unit in which ethane in the ethane stream is converted into ethylene. In an embodiment, the ethylene producing unit is a steam cracking unit. The ethane stream in line 212 may be cracked under steam in a furnace to produce a cracked stream including an ethylene stream. The ethane stream may be charged to the ethane steam cracking unit in the gas phase. The ethane steam cracking unit may preferably be operated at a temperature of about 750° C. (1382° F.) to about 950° C. (1742° F.). The cracked stream exiting the furnace of the ethane steam cracking unit may be in a superheated state. One or more quench columns, or other devices known in the art, but preferably an oil quench column and/or a water quench column, may be used for quenching or separating the cracked stream into a plurality of cracked streams. The ethane steam cracking unit may further comprise additional distillation columns, amine wash columns, compressors, expanders, etc. to separate the cracked stream into cracked streams rich in individual light olefins the most predominant of which is the ethylene stream. The ethylene stream may comprise a yield of at least 75 wt %, preferably at least 80 wt %, ethylene based on the ethane stream in line 212. Among the other components in the cracked stream exiting the ethane steam cracking, ethylene producing unit may be hydrogen, methane, propylene, butene, and pyrolysis gas. Each of these components may be recovered and further processed.

The ethylene stream and a propylene stream from the ethylene producing unit may be recovered or transported to polymerization plants, chemical plants or exported. A butene stream may be recovered and used to produce plastics or other petrochemicals by processes such as polymerization or exported. Product recovery of at least 50 wt %, typically at least 60 wt % and suitably at least 70 wt % of valuable ethylene, propylene, and butylene products is achievable from the ethane steam cracking unit based on the ethane stream in line 212.

The propane stream in line 213 may be charged to a propylene producing unit (not shown) in which propane in the propane stream is converted into propylene. The propylene producing unit may be a propane dehydrogenation (PDH) unit. PDH catalyst is used in a dehydrogenation reaction process to catalyze the dehydrogenation of propane. The conditions in the dehydrogenation reactor may include a temperature of about 500 to about 800° C., a pressure of about 40 to about 310 kPa (abs) and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 400 to about 5000 micrometers with the average diameter of about 600 to about 3500 micrometers. Preferably, the surface area of the catalyst support is about 85 to about 140 m²/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane stream perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product may include quench contacting and fractionation to produce a propylene product stream. Unreacted propane may be recycled to the dehydrogenation reactor and lighter gases may be recycled to the regenerator as fuel gas to be combusted to provide enthalpy for the reaction.

The propylene producing unit may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr⁻¹. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

Dehydrogenation effluent from the propylene producing unit 40 may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene. Propane may be recycled as feed to the propylene producing unit.

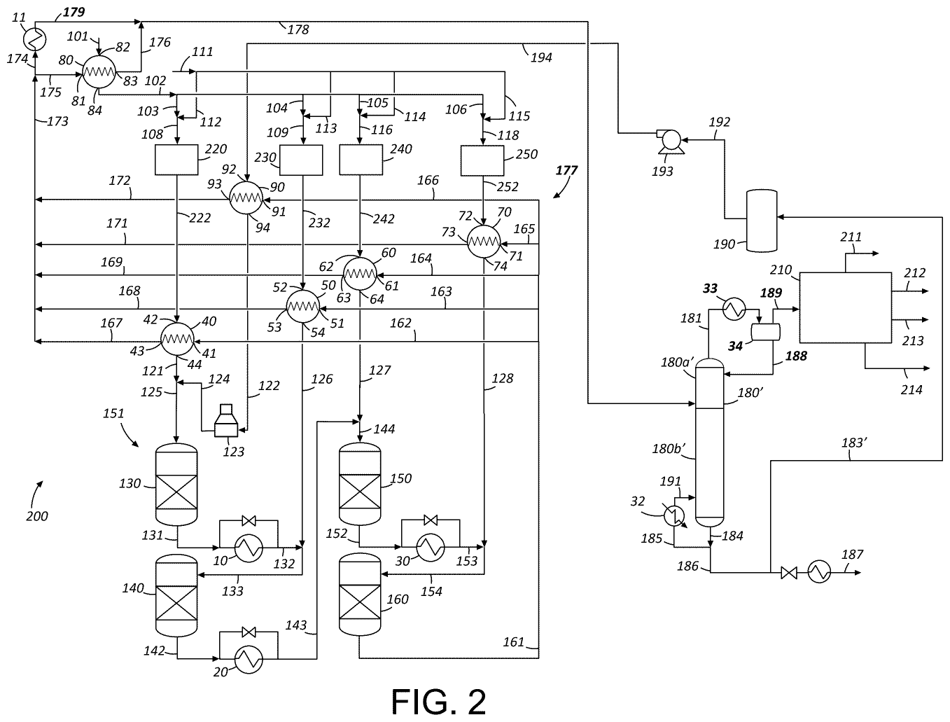

Another exemplary embodiment of the process and an apparatus for converting naphtha to ethane and propane 200 is shown in FIG. 2. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). ‘The configuration and operation of the embodiment of FIG. 2 is essentially the same as in FIG. 1 with the following exceptions.

In the embodiment shown in FIG. 2, the splitter column 180′ does not comprise an upper reboiler 31. The splitter column 180′ comprises a lower reboiler 32 for the heating requirements of the column. The splitter column 180′ comprises an upper section 180a′ and a lower section 180b′. The combined light paraffin stream in line 178 is fed to a splitter column 180′. The combined light paraffin stream in line 178 may be fed to the splitter column 180′ at a location to the upper section 180a′ of the splitter column 180′. The splitter column 180 produces an overhead stream 181 and an aromatic product stream 186. The overhead stream in line 181 may be cooled in the condenser 33 and passed to condenser receiver 34. A liquid stream 188 comprising aromatics is taken from the condenser receiver 34 and recycled to the splitter column 180′ as reflux. A vapor stream 189 comprising C4− hydrocarbons is taken from the condenser receiver and may be processed in the NEP separation unit 210 to separate ethane and propane product streams as described earlier in FIG. 1.

The bottoms aromatics stream is taken in bottoms line 184 from the splitter column 180′. A bottoms reboil stream is taken from the bottoms stream in line 185, passed through the bottoms reboiler 32 and recycled back to the splitter column 180 in the reboiled line 191. The aromatics product stream is taken in line 186 from the bottoms of the splitter column 180. In accordance with the present disclosure, the aromatic product stream in line 186 may comprise greater than 95 wt % aromatics. The aromatic product stream may be sent to storage in line 187. In the embodiment shown in FIG. 2, the aromatic recycle stream in line 183′ may be taken from the bottom aromatics product stream in line 186. The aromatic recycle stream in line 183′ is suitable to recycle to the NEP reactor 151 as the aromatics diluent stream. The rest of the process is the same as previously described for FIG. 1.

### EXAMPLES

### Example 1

The light paraffin separation of FIG. 1 was simulated using process modeling software. Table 1 provides simulated stream properties and compositions for the light paraffin stream in line 178, C4− product stream in line 189, aromatic recycle stream which was taken as a side product in line 183, and aromatic product stream in line 186.

TABLE 1

| Stream | Unit | 178 | 189 | 183 | 186 |
|---|---|---|---|---|---|
| Temperature | ° C. | 130 | 40 | 194 | 257 |
| Pressure | kPa(a) | 1718 | 1600 | 1718 | 1725 |
| Vapor Fraction | | 0.956 | 1 | 0 | 0 |
| mass flow | kg/h | 100,000 | 64,953 | 30,686 | 4,361 |
| Composition | | | | | |
| Hydrogen | wt % | 1.0% | 1.54% | 0.00% | 0.00% |
| Methane | wt % | 5.0% | 7.70% | 0.00% | 0.00% |
| Ethane | wt % | 27.0% | 41.21% | 0.76% | 0.00% |
| Propane | wt % | 27.0% | 40.51% | 2.24% | 0.01% |
| n-Butane | wt % | 5.0% | 7.18% | 1.10% | 0.05% |
| Toluene | wt % | 32.0% | 1.86% | 87.56% | 89.87% |
| Naphthalene | wt % | 3.0% | 0.00% | 8.34% | 10.07% |

The light paraffin stream in line 178 was fed to a trayed distillation column 180. The overhead vapor in line 181 was cooled to about 40° C. in the condenser 33 then sent to the receiver 34 to separate the vapor product comprising C4− in line 189 and the liquid reflux in line 188 which was returned to the top of the column. The net liquid was directed to the upper reboiler 31 and the upper reboiled stream in line 182 was returned to the column. The aromatic side product stream in line 183 was taken from the column. A bottoms reboil stream in line 185 was taken from the bottoms liquid stream in line 184. The bottoms reboil stream in line 185 was heated in a circulating lower reboiler 32, and the resulting reboiled stream in line 191 was returned to the column sump. The remaining bottoms liquid stream was discharged in line 186 and taken as product with a purity specification 0.1 wt % C4−. The aromatic side product in line 183 had greater than 95 wt % aromatics, and all or a portion of this stream is suitable to recycle to the NEP reactor 151 as the aromatics diluent stream. The condenser 33 duty for this separation was −21.3 GJ/h which may be provided by cooling water or another suitable cooling medium. The upper reboiler 31 duty was 5.2 GJ/h, and the upper reboiled stream in line 182 temperature was about 195° C. This temperature is low enough that heat for the upper reboiler 31 may be supplied by high-pressure steam typically between 30-45 bar(g). The circulating bottom reboiler 32 duty for this separation was 1.0 GJ/h which can be provided by hot oil or fired heat because the temperature of the reboiled stream in line 191 was about 259° C. which was hotter than can be supplied by HP steam typically provided between 30-45 bar(g).

### Example 2

The light paraffin separation of FIG. 2 was simulated using process modeling software. Table 2 below provides simulated stream properties and compositions for the light paraffin stream in line 178, C4− product stream in line 189, and bottom aromatic product stream in line 186.

TABLE 2

| Stream | Unit | 178 | 189 | 186 |
|---|---|---|---|---|
| Temperature | ° C. | 130 | 40 | 257 |
| Pressure | kPa(a) | 1718 | 1600 | 1738 |
| Vapor Fraction | | 0.956 | 1 | 0 |
| Mass Flow | kg/h | 100,000 | 66,181 | 33,819 |
| Composition | | | | |
| Hydrogen | wt % | 1.0% | 1.51% | 0.00% |
| Methane | wt % | 5.0% | 7.56% | 0.00% |
| Ethane | wt % | 27.0% | 40.80% | 0.00% |
| Propane | wt % | 27.0% | 40.80% | 0.00% |
| n-Butane | wt % | 5.0% | 7.50% | 0.11% |
| Toluene | wt % | 32.0% | 1.84% | 91.02% |
| Naphthalene | wt % | 3.0% | 0.00% | 8.87% |

The light paraffin stream in line 178 was fed to a trayed distillation column 180′. The overhead vapor in line 181 was cooled to about 40° C. in the condenser 33 then sent to the receiver 34 to separate the vapor product comprising C4− in line 189 and the liquid reflux in line 188 which was returned to the top of the column 180′. A bottoms reboil stream in line 185 was taken from the bottoms liquid stream in line 184. The bottoms reboil stream in line 185 was heated in a circulating lower reboiler 32 and the resulting reboiled stream in line 191 was returned to the column sump. The remaining bottoms liquid stream was discharged in line 186 and taken as product with a purity specification 0.1 wt % C4−. The aromatics product in line 186 had greater than 99 wt % aromatics, and all or a portion of this stream is suitable to recycle to the NEP reactor 151 as the aromatics diluent stream. The condenser 33 duty for this separation was −21.8 GJ/h which may be provided by cooling water or another suitable cooling medium. The bottom reboiler 32 duty for this separation was 11.0 GJ/h which can be provided by hot oil or fired heat because the temperature of the reboiled stream in line 191 was about 259° C. which is hotter than can be supplied by HP steam typically provided between 30-45 bar(g).

### SPECIFIC EMBODIMENTS

While the following is described in conjunction with specific embodiments, it will be understood that this description is intended to illustrate and not limit the scope of the preceding description and the appended claims.

A first embodiment of the present disclosure is a process for converting naphtha to ethane and propane, comprising contacting a naphtha stream and a hydrogen stream with a catalyst in a reactor to produce a converted stream; separating the converted stream into a light paraffin stream and an aromatic stream; and recycling the aromatic stream to the reactor; and separating the light paraffin stream into an ethane stream and a propane stream. An embodiment of the present disclosure is one, any or all of prior embodiments in this paragraph up through the first embodiment in this paragraph wherein the aromatic stream is taken from a side of a fractionation column. An embodiment of the present disclosure is one, any or all of prior embodiments in this paragraph up through the first embodiment in this paragraph wherein the aromatic stream predominantly comprises single ring aromatics. An embodiment of the present disclosure is one, any or all of prior embodiments in this paragraph up through the first embodiment in this paragraph wherein the fractionation column comprises an upper reboiler located at a stage above the side stream. An embodiment of the present disclosure is one, any or all of prior embodiments in this paragraph up through the first embodiment in this paragraph wherein the upper reboiler is heated with steam. An embodiment of the present disclosure is one, any or all of prior embodiments in this paragraph up through the first embodiment in this paragraph wherein the ratio of aromatic stream to naphtha stream is in the range of about 0.1 to about 1.0 by weight. An embodiment of the present disclosure is one, any or all of prior embodiments in this paragraph up through the first embodiment in this paragraph wherein the fractionation column produces an aromatic product stream comprising at least 95 wt % aromatics.

An embodiment of the present disclosure is one, any or all of prior embodiments in this paragraph up through the first embodiment in this paragraph wherein the aromatic stream is recycled to a first reactor of a plurality of reactors. An embodiment of the present disclosure is one, any or all of prior embodiments in this paragraph up through the first embodiment in this paragraph further comprising heating the aromatic stream with the converted stream to preheat the aromatics stream and cool the converted stream. An embodiment of the present disclosure is one, any or all of prior embodiments in this paragraph up through the first embodiment in this paragraph wherein the converted stream is a final converted stream of the plurality of reactors. An embodiment of the present disclosure is one, any or all of prior embodiments in this paragraph up through the first embodiment in this paragraph further comprising separating the naphtha stream into a plurality of naphtha streams; separating a hydrogen stream into a plurality of hydrogen streams; mixing each of the plurality of naphtha streams with a dedicated hydrogen stream from the plurality of hydrogen streams to provide a plurality of charge streams; and passing each of the plurality of charge streams to a dedicated one of a plurality of reactors to produce a plurality of converted streams; and separating one of the plurality of converted streams into the ethane stream and the propane stream. An embodiment of the present disclosure is one, any or all of prior embodiments in this paragraph up through the first embodiment in this paragraph further comprising vaporizing each of the plurality of charge streams to produce a plurality of vaporized charge streams; and contacting each of the plurality of vaporized charge streams with a catalyst in the dedicated one of a plurality of reactors to produce the plurality of converted streams. An embodiment of the present disclosure is one, any or all of prior embodiments in this paragraph up through the first embodiment in this paragraph wherein the plurality of charge streams is heated with a converted stream from a last reactor.

A second embodiment of the present disclosure is a process for converting naphtha to ethane and propane, comprising combining a naphtha stream and a hydrogen stream to provide a charge stream, vaporizing the charge stream to provide a vaporized charge stream; contacting the vaporized charge stream with a catalyst in a reactor to produce a converted stream; separating the converted stream into a light paraffin stream and an aromatic stream; and recycling the aromatic stream to the reactor; and separating the light paraffin stream into an ethane stream and a propane stream. An embodiment of the present disclosure is one, any or all of prior embodiments in this paragraph up through the second embodiment in this paragraph further comprising heating the vaporized charge stream to provide a heated charge stream; and passing a heated charge stream to the reactor. An embodiment of the present disclosure is one, any or all of prior embodiments in this paragraph up through the second embodiment in this paragraph wherein the aromatic stream is a side stream from a fractionation column. An embodiment of the present disclosure is one, any or all of prior embodiments in this paragraph up through the second embodiment in this paragraph wherein the aromatic stream predominantly comprises single ring aromatics.

A third embodiment of the present disclosure is an apparatus for converting naphtha, comprising a naphtha feed line; a hydrogen feed line; a plurality of reactors, wherein each reactor of the plurality of reactors is in fluid communication with the naphtha feed line and the hydrogen feed line; and a fractionation column in downstream fluid communication with the plurality of reactors, the fractionation column comprising an upper reboiler and a lower reboiler. An embodiment of the present disclosure is one, any or all of prior embodiments in this paragraph up through the third embodiment in this paragraph wherein each reactor of the plurality of reactors is in fluid communication with a branch naphtha line of the naphtha feed line. An embodiment of the present disclosure is one, any or all of prior embodiments in this paragraph up through the third embodiment in this paragraph wherein each reactor of the plurality of reactors is in fluid communication with a branch hydrogen line of the hydrogen feed line. An embodiment of the present disclosure is one, any or all of prior embodiments in this paragraph up through the third embodiment in this paragraph, wherein the fractionation column comprises a side line in fluid communication with the multistage reactor. An embodiment of the present disclosure is one, any or all of prior embodiments in this paragraph up through the third embodiment in this paragraph further comprising a plurality of heat exchangers, wherein a hot inlet of a heat exchanger is in fluid communication with an upstream reactor and a hot outlet of the heat exchanger is in fluid communication with a downstream reactor. An embodiment of the present disclosure is one, any or all of prior embodiments in this paragraph up through the third embodiment in this paragraph wherein a hot inlet of a heat exchanger is in fluid communication with a final reactor of the plurality of reactors, and a cold inlet of a heat exchanger is in fluid communication with a branch naphtha line. An embodiment of the present disclosure is one, any or all of prior embodiments in this paragraph up through the third embodiment in this paragraph further comprising an aromatics surge drum in downstream fluid communication with the fractionation column and in upstream fluid communication with a first reactor of the plurality of reactors.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting naphtha to ethane and propane, comprising:
contacting a naphtha stream and a hydrogen stream with a catalyst in a reactor to produce a converted stream;
separating the converted stream into a light paraffin stream and an aromatic stream; and
recycling the aromatic stream to the reactor; and
separating the light paraffin stream into an ethane stream and a propane stream.

2. The process of claim 1 wherein the aromatic stream is taken from a side of a fractionation column.

3. The process of claim 2 wherein the aromatic stream predominantly comprises single ring aromatics.

4. The process of claim 2 wherein the fractionation column comprises an upper reboiler located at a stage above the side stream.

5. The process of claim 4 wherein the upper reboiler is heated with steam.

6. The process of claim 1 wherein the ratio of aromatic stream to naphtha stream is between 0.1 to 1.0 by weight.

7. The process of claim 1 wherein the fractionation column produces an aromatic product stream comprising at least 95 wt % aromatics.

8. The process of claim 1 wherein the aromatic stream is recycled to a first reactor of a plurality of reactors.

9. The process of claim 1 further comprising:
heating the aromatic stream with the converted stream to preheat the aromatic stream and cool the converted stream.

10. The process of claim 9 wherein the converted stream is a final converted stream of the plurality of reactors.

11. The process of claim 1 further comprising:
separating said naphtha stream into a plurality of naphtha streams;
separating a hydrogen stream into a plurality of hydrogen streams;
mixing each of said plurality of naphtha streams with a dedicated hydrogen stream from said plurality of hydrogen streams to provide a plurality of charge streams; and
passing each of said plurality of charge streams to a dedicated one of a plurality of reactors to produce a plurality of converted streams; and
separating one of said plurality of converted streams into said ethane stream and said propane stream.

12. The process of claim 11 further comprising:
vaporizing each of said plurality of charge streams to produce a plurality of vaporized charge streams; and
contacting each of said plurality of vaporized charge streams with a catalyst in said dedicated one of a plurality of reactors to produce said plurality of converted streams.

13. The process of claim 12 wherein said plurality of charge streams is heated with a converted stream from a last reactor.

14. An apparatus for converting naphtha, comprising:
a naphtha feed line;
a hydrogen feed line;
a plurality of reactors, wherein each reactor of the plurality of reactors is in fluid communication with the naphtha feed line and the hydrogen feed line; and
a fractionation column in downstream fluid communication with the plurality of reactors, the fractionation column comprising an upper reboiler and a lower reboiler.

15. The apparatus of claim 14 wherein each reactor of the plurality of reactors is in fluid communication with a branch naphtha line of the naphtha feed line.

16. The apparatus of claim 14 wherein each reactor of the plurality of reactors is in fluid communication with a branch hydrogen line of the hydrogen feed line.

17. The apparatus of claim 14, wherein the fractionation column comprises a side line in fluid communication with one of the plurality of reactors.

18. The apparatus of claim 14 further comprising a plurality of heat exchangers, wherein a hot inlet of a heat exchanger is in fluid communication with an upstream reactor and a hot outlet of the heat exchanger is in fluid communication with a downstream reactor.

19. The apparatus of claim 18, wherein a hot inlet of a heat exchanger is in fluid communication with a final reactor of the plurality of reactors, and a cold inlet of a heat exchanger is in fluid communication with a branch naphtha line.

20. The apparatus of claim 14 further comprising an aromatics surge drum in downstream fluid communication with the fractionation column and in upstream fluid communication with a first reactor of the plurality of reactors.
